SEQUENCE LISTING (8 sequences)

SEQ ID NO: 1 - protein, 520 aa, Norovirus
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALE

SEQ ID NO: 2 - protein, 517 aa, Sapovirus
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 3 - protein, 533 aa, Vesivirus
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLE

SEQ ID NO: 4 - protein, 526 aa, Artificial
  Norovirus-RdRp having his tag
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALEHHHHHH

SEQ ID NO: 5 - protein, 523 aa, Artificial
  Sapovirus-RdRp having his tag
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEMEHHHHHH

SEQ ID NO: 6 - protein, 539 aa, Artificial
  Vesivirus-RdRp having his tag
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLEHHHHHH

SEQ ID NO: 7 - RNA, 24 nt, Artificial
  single stranded RNA template strand ssRX21 for
gcugaugccgucaaguuuaccccc

SEQ ID NO: 8 - RNA, 24 nt, Artificial
  single stranded antisense product strand ssRX21 rev
ggggguaaacuugacggcaucagc

(19) United States
(12) Patent Application Publication
(10) Pub. No.: US 2011/0053226 A1
(43) Pub. Date: Mar. 3, 2011

(54) METHOD FOR ENZYMATIC SYNTHESIS OF CHEMICALLY MODIFIED RNA

(75) Inventor: Jacques Rohayem, Dresden (DE)

(73) Assignee: RiboxX GmbH, Radebeul (DE)

(21) Appl. No.: 12/864,764

(22) PCT Filed: Jun. 9, 2009
(86) PCT No.: PCT/EP2009/057119

§ 371 Date: Jul. 27, 2010

(30) Foreign Application Priority Data
Jun. 13, 2008 (EP) 08010829.3

(51) Int. Cl. C12P 19/34; C12N 9/12

(52) U.S. Cl. 435/91.21; 435/91.3; 435/194

(57) ABSTRACT

The present invention relates to a method for enzymatically synthesizing chemically modified RNA by using RNA-dependent RNA polymerases (RdRp), especially RdRps from viruses of the Caliciviridae family. The method of the present invention is particularly useful for preparing RNA molecules of increased stability especially with respect to RNA degradation, for example for in vivo applications. Further subject matter of the present invention relates to a kit for carrying out the enzymatic synthesis of the chemically modified RNA.

The present invention relates to a method for enzymatically synthesizing chemically modified RNA by using RNA-dependent RNA polymerases (RdRp), especially RdRps from viruses of the Caliciviridae family. The method of the present invention is particularly useful for preparing RNA molecules of increased stability especially with respect to RNA degradation, for example for in vivo applications. Further subject matter of the present invention relates to a kit for carrying out the enzymatic synthesis of the chemically modified RNA.

RNA molecules containing chemically modified ribonucleotides are useful for various applications in science and medicine. In particular, it is generally known that especially backbone-modified RNAs possess an increased stability, e.g. with respect to degradation by RNases, which is of importance for in vivo applications, for example in the case of antisense ribonucleotides as well as siRNAs (small interfering RNAs) for RNA interference (RNAi).

Hitherto, such modified RNA is generally prepared by solid-phase chemical synthesis methods. However, such chemical methods have several drawbacks, for example they are expensive and time consuming. Those drawbacks are an important limitation for the broad diffusion of the RNAi-therapy. Indeed, it is predicted that several tons of RNAi-molecules may be needed each year for the treatment of so far untreatable diseases such as cancer, viral infectious diseases and degenerative diseases (i.e. acute macular degeneration). Those so far untreatable diseases may extensively benefit from RNAi-Therapy.

Accordingly, there is a substantial need for alternative methods for the production of chemically modified RNA molecules, in particular for therapeutic applications. It is known from WO-A-2007/012329 that viruses of the Caliciviridae family can be used for amplification and/or labelling of RNA molecules. In this disclosure, the authors also describe that the calicivirus RdRps can make use of fluorescently coupled, biotin-coupled or radioactively-coupled NTPs used for labelling of the RNA. However, concrete examples that the calicivirus RdRps can factually incorporate such coupled NTPs are completely missing. Furthermore, it is clear that the term NTP analogue in WO-A-2007/012329 is exclusively directed to such analogues that can be used for the detection of the labelled RNA by fluorescence emission, radioactive emission and/or by providing a partner for a chemical or biological binding pair such as for example biotin. Thus, the incorporation of NTP-analogues as envisaged in WO-A-2007/012329 is exclusively directed to the detection of coupled NTPs used for detection of RNA molecules labelled with such NTP-analogues.

Furthermore, another group suggested recently the use of chemically modified ribonucleotides for the inhibition of a norovirus RdRp (Zamyatkin et al. (2008) J. Biol. Chem. 283: 7705-7712).

The solution to this technical problem is provided by the embodiments of the present invention as defined in the claims.

In particular, the present invention provides a method for enzymatically synthesizing chemically modified RNA comprising the steps of:
(a) providing ssRNA template (single-stranded RNA template); and
(b) interaction of the ssRNA template with a protein having RNA-dependent RNA polymerase (RdRp) activity under conditions sufficient for RNA synthesis in the presence of at least one ribonucleotide triphosphate having chemical modification at the ribose, phosphate and/or base moiety to provide chemically modified double-stranded RNA (dsRNA), wherein the chemical modification does not result in labelling of the dsRNA with a radioactive, fluorescent and/or chemical label used for detection of said dsRNA.

In view of the prior art it is highly surprising that RdRps, especially corresponding enzymes from the calicivirus family (Caliciviridae) can factually incorporate diverse chemically modified ribonucleotides into dsRNA by synthesizing a complementary strand to a single stranded RNA (ssRNA) template.

It is further contemplated according to the present invention that the incorporation of chemically modified ribonucleoside triphosphates into RNA will be used for the large-scale industrial production of such modified RNA. Thus, it is preferred that chemically modified ribonucleoside triphosphates are used in an amplification reaction which comprises the further steps:
(c) separating the dsRNA into ssRNA, and optionally performing the following steps (d) to (f):
(d) repeating steps (b) and (c) n times with n being an integer of at least 1;
(e) carrying out a final step (b); and
(f) stopping the reaction.

Preferably, n is an integer from 10 to 50, more preferred 15 to 40, most preferred 20 to 30.

The strand separation according step (c) may be carried out by application of heat (heat denaturation), by chemical denaturation or enzymatically, preferably by a helicase. Especially in the case of heat denaturation, it is desirable to add further RdRp molecules to the reaction mixture for performing the next step (b).

It is further preferred that the protein having RdRp activity has a “right hand conformation” and that the amino acid sequence of said protein comprises a conserved arrangement of the following sequence motifs:

a. XXDYS
b. GXPSG
c. YGDD
d. XXYGL
e. XXXXFLXRXX

with the following meanings:
D: aspartate
Y: tyrosine
S: serine
G: glycine
P: proline
L: leucine
F: phenylalanine
R: arginine
X: any amino acid.

The so-called “right hand conformation” as used herein means that the tertiary structure (conformation) of the protein folds like a right hand with finger, palm and thumb, as observed in most template-dependent polymerases

The sequence motif “XXDYS” is the so-called A-motif. The A-motif is responsible for the discrimination between ribonucleosides and deoxyribonucleosides. The motif “GXPSG” is the so-called B-motif. The B-motif is conserved within all representatives of this RdRp family of the corresponding polymerases from the Caliciviridae. The motif “YGDD” (“C-motif) represents the active side of the enzyme. This motif, in particular the first aspartate residue (in bold, YGDD) plays an important role in the coordination of the metal ions during the Mg²⁺/Mn²⁺ dependent catalysis. The motif “XXYGL” is the so-called D-motif. The D-motif is a feature of template-dependent polymerases. Finally, the “XXXXFLXRXX” motif (E-motif) is a feature of RNA-dependent RNA polymerases which discriminates them from DNA-dependent RNA polymerases.

Typical representatives of the above types of RdRps are the corresponding enzymes of the calicivirus family (Caliciviridae). The RdRps of the calicivirus family are capable of synthesizing complementary strands using as a template any ssRNA template in vitro, including heterologous viral, eukaryotic and prokaryotic templates. The ssRNA template may be positive stranded or negative stranded.

The above-defined protein having RdRp activity is capable of synthesizing a complementary on a strand both by elongation of a corresponding primer and by novo synthesis of a complementary strand in the absence of a primer. The primer, if desired, may be a sequence specific primer or may be a random primer or may be an oligo-T-primer or an oligo-U-Primer. More details of the characteristic features of the calicivirus RdRp can be found in WO-A-2007/012329

Preferably, the RNA-dependent RNA-polymerase is an RdRp of a human and/or non-human pathogenic calicivirus. Especially preferred is an RdRp of a norovirus, sapovirus, vesivirus or lagovirus, for example the RdRP of the norovirus strain HuCV/NL/Dresden174/1997/GE (GenBank acc. No AY741811) or of the sapovirus strain pJG-Sap01 (GenBank acc. No AY694184) or an RNA-dependent RNA polymerase of the vesivirus strain FCV/Dresden/2006/GE (GeBank acc. No DQ424892).

According to especially preferred embodiments of the invention the RdRp is a protein having an amino acid sequence according SEQ ID NO: 1 (norovirus-RdRP), SEQ ID NO: 2 (sapovirus-RdRP) or SEQ ID NO: 3 (vesivirus-RdRP). The person skilled in the art is readily capable of preparing such RdRP, for example by recombinant expression using suitable expression vectors and host organisms (cf. WO-A-2007/012329). To facilitate purification of the RdRp in recombinant expression, it is preferred that the RdRp is expressed with a suitable tag (for example GST or (His)-6-tag) at the N- or C-terminus of the corresponding sequence. For example, a histidine tag allows the purification of the protein by affinity chromatography over a nickel or cobalt column in a known fashion. Examples of embodiments of RdRps fused to a histidine tag are the proteins having an amino acid sequence according to SEQ ID NO: 4, SEQ ID NO: 5 or SEQ ID NO: 6. SEQ ID NO: 4 corresponds to a norovirus-RdRP having a histidine tag. SEQ ID NO: 5 corresponds to the amino acid sequence of a sapovirus-RdRP having a histidine tag. SEQ ID NO: 6 corresponds to the amino acid sequence of vesirius-RdRP having a histidine tag.

SEQ ID NO: 1:
MGGDSKGTYCGAPILGPGSAPKLSTKTKFWRSSTTPLPPGTYEPAYLG
GKDPRVKGGPSLQQVMRDQLKPFTEPRGKPPKPSVLEAAKKTIINVLE
QTIDPPEKWSFTQACASLDKTTSSGHPHHMRKNDCWNGESFTGKLADQ
ASKANLMFEGGKNMTPVYTGALKDELVKTDKIYGKIKKRLLWGSDLAT
MIRCARAFGGLMDELKAHCVTLPIRVGMNMNEDGPIIFERHSRYKYHY
DADYSRWDSTQQRAVLAAALEIMVKFSSEPHLAQVVAEDLLSPSVVDV
GDPKISINEGLPSGVPCTSQWNSIAHWLLTLCALSEVTNLSPDIIQAN
SLFSFYGDDEIVSTDIKLDPEKLTAKLKEYGLKPTRPDKTEGPLVISE
DLNGLTFLRRTVTRDPAGWFGKLEQSSILRQMYWTRGPNHEDPSETMI
PHSQRPIQLMSLLGEAALHGPAFYSKISKLVIAELKEGGMDFYVPRQE
PMFRWMRFSDLSTWEGDRNLAPSFVNEDGVEVDKLAAALE
SEQ ID NO: 2:
MKDEFQWKGLPVVKSGLDVGGMPTGTRYHRSPAWPEEQPGETHAPAPF
GAGDKRYTFSQTEMLVNGLKPYTEPTAGVPPQLLSRAVTHVRSYIETI
IGTHRSPVLTYHQACELLERTTSCGPPVQGLKGDYWDEEQQQTYGVLA
NHLSQAWDKANKGIAPRNAYKLALKDELRPIEKNKAGKRRLLWGCDAA
TTLIATAAFKAVATRLQVVTPMTPVAVGINMDSVQMQVMNDSLKGGVL
YCLDYSKWDSTQNPAVTAASLAILERFAEPHPIVSCAIEALSSPAEGY
VNDIKFVTRGGLPSGMPFTSVVNSINHMIYVAAAILQAYESHNVPYTG
NVPQVETVHTYGDDCMYSVCPATASIFHAVLANLTSYGLKPTAADKSD
AIKPTNTPVFLKRTFTQTPHGVRALLDITSITRQFYWLKANRTSDPSS
PPAFDRQARSAQLENALAYASQHGPVVFDTVRQIAIKTAQGEGLVLVN
TNYDQALATYNAWFIGGTVPDPVGHTEGTHKIVFEME
SEQ ID NO: 3:
MKVTTQKYDVTKPDISYKGLICKQLDEIRVIPKGTRLHVSPAHTDDYD
ECSHQPASLGSGDPRCPKSLTAIVVDSLKPYCEKTDGPPHDILHRVQR
MLIDHLSGFVPMNISSEPSMLAAFHKLNHDTSCGFYLGGRKKDHMIGG
EPDKPLLDLLSSKWKLATQGIGLPHEYTIGLKDELRPVEKVQEGKRRM
IWGCDVGVATVCAAAFKGVSDAITANHQYGPVQVGINMDGPSVEALYQ
RIRSAAKVFAVDYSKWDSTQSPRVSAASIDILRYFSDRSPIVDSAANT
LKSPPIAIFNGVAVKVTSGLPSGMPLTSVINSLNHCLYVGCAILQSLE
SRNIPVTWNLFSTFDMMTYGDDGVYMFPMMFASVSDQIFANLTAYGLK
PTRVDKSVGAIEPIDPESVVFLKRTITRTPHGIRGLLDRGSIIRQFYY
IKGENSDDWKTPPKTIDPTSRGQQLWNACLYASQHGPEFYNKVYRLAE
KAVEYEELHFEPPSYHSALEHYNNQFNGVDTRSDQIDASVMTDLHCDV
FEVLE
SEQ ID NO: 4:
MGGDSKGTYCGAPILGPGSAPKLSTKTKFWRSSTTPLPPGTYEPAYLG
GKDPRVKGSPSLQQVMRDQLKPFTEPRGKPPKPSVLEAAKKTIINVLE
QTIDPPEKWSFTQACASLDKTTSSGHPHHMRKNDCWNGESFTGKLADQ
ASKANLMFEGGKNMTPVYTGALKDELVKTDKIYGKIKKRLLWGSDLAT
MIRCARAFGGLMDELKAHCVTLPIRVGMNMNEDGPIIFERHSRYKYHY
DADYSRWDSTQQRAVLAAALEIMVKFSSEPHLAQVVAEDLLSPSVVDV
GDFKISINSGLPSGVPCTSQWNSIAHWLLTLCALSEVTNLSPDIIQAN
SLFSFYGDDEIVSTDIKLDPEKLTAKLKEYGLKPTRPDKTEGPLVISE
DLNGLTFLRRTVTRDPAGWFGKLEQSSILRQMYWTRGPNHRDPSETMI
PHSQRPIQLMSLLGEAALHGPAFYSKISKLVIAELKEGGMDFYVPRQE
PMFRWMRFSDLSTWEGDRNLAPSFVNEDGVEVDKLAAALEHHHHHH
SEQ ID NO: 5:
MKDEFQWKGLFVVKSGLDVGGMPTGTRYHRSPAWPEEQPGETHAPAPF
GAGDKRYTFSQTEMLVNGLKPYTEPTAGVPPQLLSRAVTHVRSYIETI
IGTHRSPVLTYHQACELLERTTSCGPFVQGLKGDYWDEEQQQYTGVLA
NHLEQAWDKANKGIAPRNAYKLALKDELRPIEKNKAGKRRLLWGCDAA
TTLIATAAFKAVATRLQVVTPMTPVAVGINMDSVQMQVMNDSLKGGVL
YCLDYSAWDSTQNPAVTAASLAILERFAEPHPIVSCAIEALSSPAEGY
VNDIKFVTRGGLPSGMPFTSVVNSINHMIYVAAAILQAYESHNVPYTG
NVFQVETVHTYGDDCMYSVCPATASIFHAVLANLTSYGLKPTAADKSD
AIKPTNTPVFLKRTFTQTPHGVRALLDITSITRQFYWLKANRTSDPSS
PPAFDRQARSAQLENALAYASQHGPVVFDTVRQIAIKTAQGEGLVLVN
TNYDQALATYNAWFIGGTVPDPVGHTEGTHKIVFEMEHHHHHH
SEQ ID NO: 6:
MKVTTQKYDVTKPDISYKGLICKQLDEIRVIPKGTRLHVSPAHTDDYD
ECSHQPASLGSGDPRCPKSLTAIVVDSLKPYCEKTDGPPHDILHRVQR
MLIDHLSGFVPMNISSEPSMLAAFHKLNMDTSCGPYLGGRKKDHMIGG
EPDKPLLDLLSSKWKLATQGIGLPHEYTIGLKDELRPVEKVQEGKRRM
IWGCDVGVATVCAAAFKGVSDAITANHQYGPVQVGINMDGPSVEALYQ
RIRSAAKVFAVDYSKWDSTQSPRVSAASIDILRYFSDRSPIVDSAANT
LKSPPIAIFNGVAVKVTSGLPSGMPLTSVINSLNHCLYVGCAILQSLE
SRNIPVTWNLFSTFDMMTYGDDGVYMFPMMFASVSDQIFANLTAYGLK
PTRVDKSVGAIEPIDPESVVFLKRTITRTPHGIRGLLDRGSIIRQFYY
IKGENSDDWKTPPKTIDPISRGQQLWNACLYASQHGPEFYNKVYRLAE
KAVEYEELHFRPPSYHSALEHYNNQFNGVDTRSDQIDASVMTDLHCDV
FEVLEHHHHHH

The method of the present invention is particularly useful for providing short RNA molecules for gene silencing applications, either by antisense technology or RNA interference.

Therefore, the ssRNA template to be used in the method of the present invention has preferably a length of 8 to 45 nucleotides, preferably of 15 to 30 nucleotides, preferably of 21 to 28 nucleotides, more preferably of 21 to 23 nucleotides. The molecules of the latter length are particularly useful for siRNA applications. The chemically modified RNA products of the methods of the present invention preferably have an increased stability as compared to the non-modified dsRNA analogues.

Especially for this purpose, the chemical modification of the at least one modified ribonucleoside triphosphate to be incorporated by the RdRp activity into the complementary strand can have a chemical modification(s) at the ribose, phosphate and/or base moiety. With respect to molecules having an increased stability, especially with respect to RNA degrading enzymes, modifications at the backbone, i.e. the ribose and/or phosphate moieties, are especially preferred.

Preferred examples of ribose-modified ribonucleoside triphosphates are analogues wherein the 2′-OH group is replaced by a group selected from H, OR, R, halo, SH, SR, NH₂, NHR, NR₂ or CN with R being C₁-C₆ alkyl, alkenyl or alkynyl and halo being F, CI, Br or I. It is clear in the context of the present invention, that the term “modified ribonucleoside triphosphate” or “modified ribonucleotide” also includes 2′-deoxy derivatives which may at several instances also be termed “deoxynucleotides”.

Typical examples of such ribonucleotide analogues with a modified ribose at the 2′ position include 2′-O-methyl-cytidine-5′-triphosphate, 2′-amino-2′-deoxy-uridine, 2′-azido-2′-deoxy-uridine-5′-triphosphate, 2′-fluoro-2′-deoxy-guanosine-5′-triphosphate and 2′-O-methyl-5-methyl-uridine-5′-triphosphate.

Examples of ribonucleoside triphosphates leading to a phosphate backbone modification in the desired dsRNA product are phosphothioate analogues.

According to the present invention, the at least one modified ribonucleoside trisphophate may also be selected from analogues having a chemical modification at the base moiety. Examples of such analogues include 5-aminoallyl-uridine-5′-triphosphate, 6-aza-uridine-5′-triphosphate, 8-aza-adenosine-5′-triphosphate, 5-bromo-uridine-5′-triphosphate, 7-deaza-adenosine-5′-triphosphate, 7-deaza-guanosine-5′-triphosphate, N⁶-methyl-adenosine-5′-triphosphate, 5-methyl-cytidine-5′-triphosphate, pseudo-uridine-5′-triphosphate, and 4-thio-uridine-5′-triphosphate.

The above and other chemically modified ribonucleoside triphosphates are commercially available, for example from Sigma-Aldrich Chemie GmbH, Munich, Germany or Trilink technologies, USA

According to the present invention, the term “conditions sufficient for RNA synthesis” means the conditions, in particular relating to buffer, temperature, salt and metal ion (if applicable) conditions that allow the RdRp to synthesise an RNA strand complementary to a template strand. Appropriate buffer, salt, metal ion, reducing agent (if applicable) and other conditions of RdRps are known to the skilled person. With regard to the RdRPs of caliciviruses, it is referred to WO-A-2007/012329. Thus, the ssRNA template is used in amounts of, e.g. 1 microgram to 4 microgram per 50 microliter reaction volume. The concentration of the ribonucleoside triphosphates (including the modified ribonucleoside trisphosphate (s)) is preferably in the range of from 0.1 micromol/l to 1 micromol/l, for example 0.4 micromol/l. The concentration of the RdRp may be for example 1 micromol/l to 10 micromol/l.

Typical buffer conditions are 10 to 80 mM, more preferred 20 to 50 mM HEPES, pH 7.0-8.0, 1 to 5 mM, for example 3 mM magnesium acetate, magnesium chloride, manganese acetate or manganese chloride and 1 to 5 mM of a reducing agent, for example DTT.

A typical stop solution contains 2 to 10 mM, preferably 4 to 8 mM ammonium acetate, and 50 to 200 mM, for example 150 mM EDTA.

Short RNA templates (e.g. as defined above) are usually prepared by chemical synthesis of two separate strands that are then annealed in vitro using an annealing buffer containing for example Tris-Hcl 50 mM, pH 8.0, NaCl 200 mM, EDTA 1 mM. Other methods for providing the ssRNA template include enzymatic manipulations, for example transcription initiation depending on selected sequences (so called “promotor”) or using a specific primer and RNA synthesis by DNA-dependent RNA polymerases following degradation of the DNA strand.

Preferred reaction volumes range from 20 to 200 microliter, preferably 50 to 100 microliter. Typically, the buffer conditions and other conditions as outlined above are provided by mixing appropriate stock solutions (usually 5× or 10× concentrated), adding the RdRPl and the ssRNA template and double distilled or deionised water to the desired final reaction volume.

A further subject matter of the present invention is a kit for carrying out the inventive method. The kit of the present invention comprises a protein having RdRp activity (preferred examples are defined above), ribonucleotides (rATP, rGTP, rCTP, rUTP, preferably as stock solutions), at least one chemically modified ribonucleoside triphosphate (as defined above and preferably selected from the examples outlined above), an appropriate buffer (preferably as a stock solution as defined above), and, optionally, a stop solution (preferably a stop solution as defined above, more preferred in the form of a 5× or 10× stock solution) and, also optionally, a helicase.

The figures show:

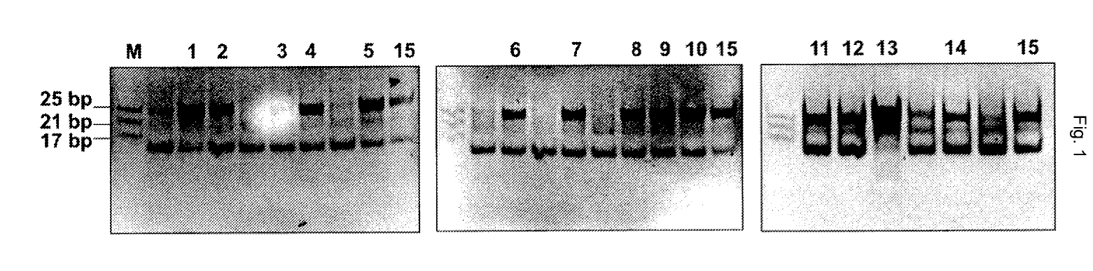

FIG. 1 shows photographic representations of polyacrylamid gel electrophoretic analysis after ethidium bromide staining of double-stranded RNA with or without modified nucleotides synthesized using calicivirus RdRp. M, molecular size marker for double-stranded RNA (Ambion/Applied Biosystems, Foster City, Calif., USA) is indicated. Single-stranded RNA template is the band below the 17 bp band of the marker M. The dsRNA product synthesized by the Calicivirus RdRp is visible at around 25 bp band of the marker M.

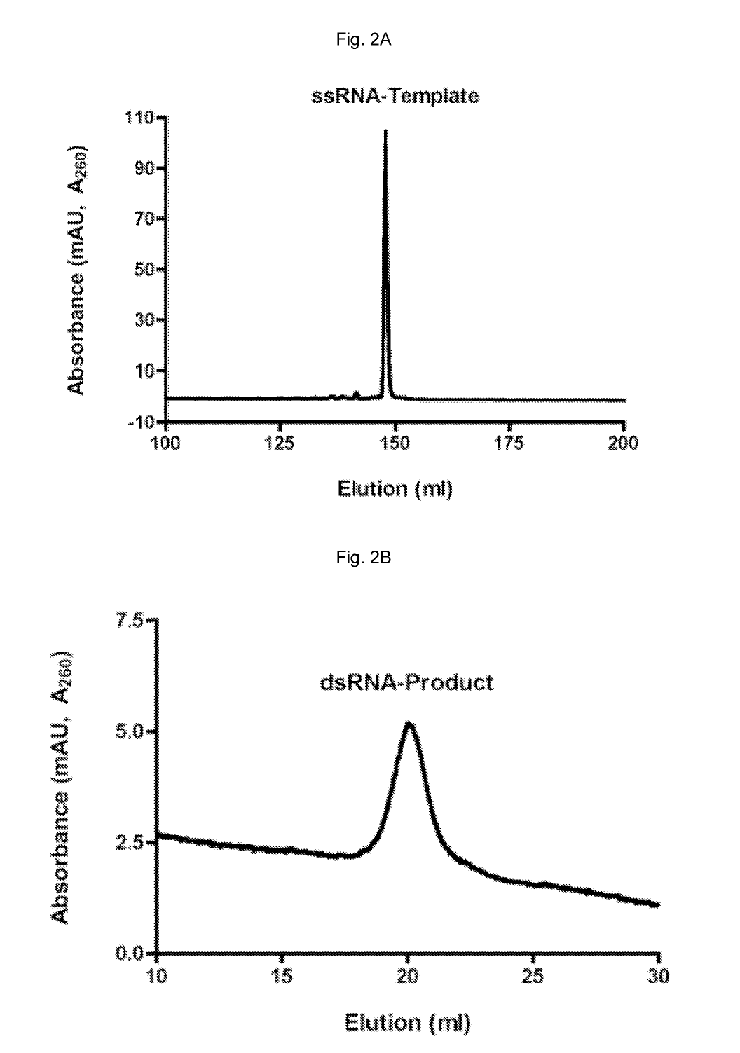

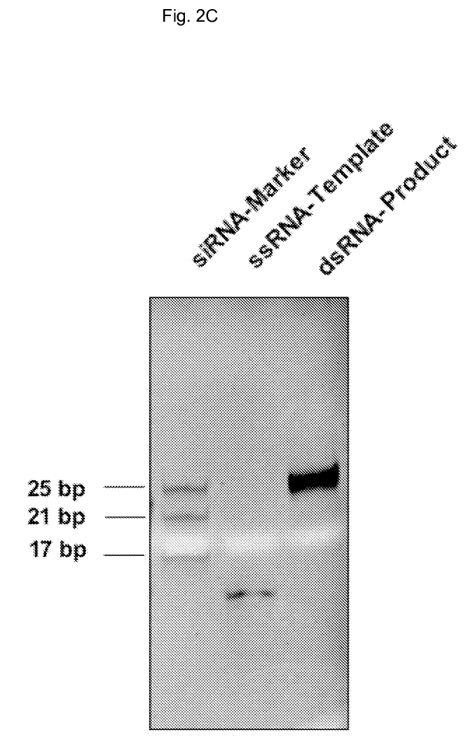

FIG. 2a shows graphical representations of elution profiles resulting in purification of the single-stranded RNA template used for enzymatic synthesis with calicivirus RdRp.

FIG. 2b shows graphical representations of elution profiles resulting in purification of the double-stranded RNA products of the enzymatic synthesis using calicivirus RdRp. The product of a reaction for a template ssRNA was purified from the reaction mixture by ion exchange chromatography and fractionized.

FIG. 2c shows a photographical representation of a polyacrylamid gel electrophoresis of the fractions indicated (marked with a dashed box) in FIG. 2b. M, molecular size marker for double-stranded RNA (Ambion/Applied Biosystem) as indicated. The template ssRNA migrates with the 17 bp marker fragment. The product of the synthesis by the calicivirus RdRp can also be seen as a band of approximately 25 bp.

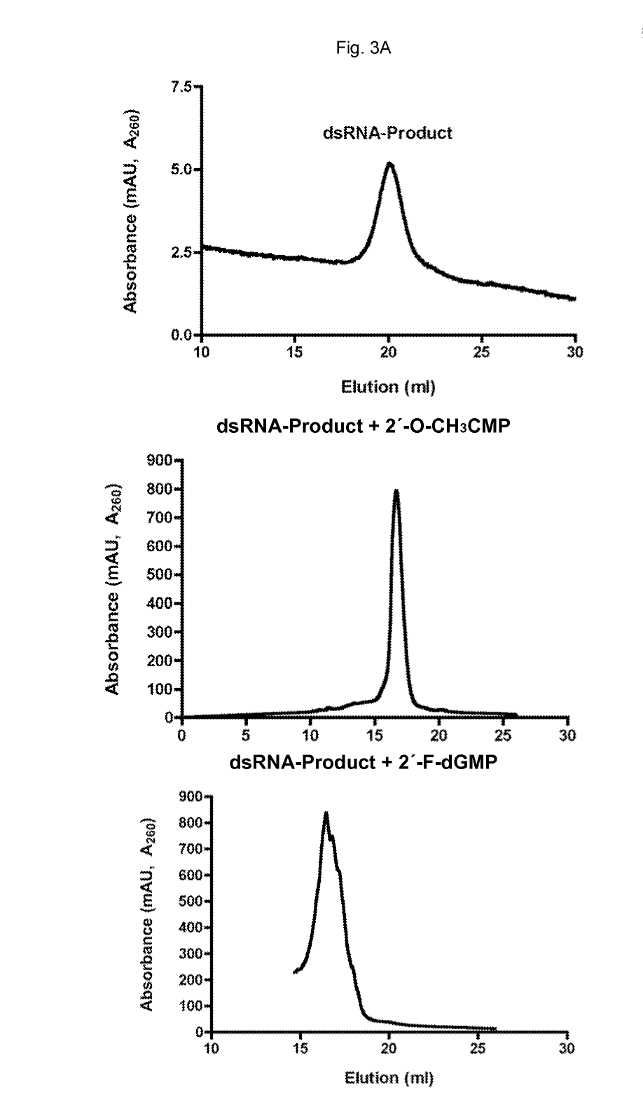

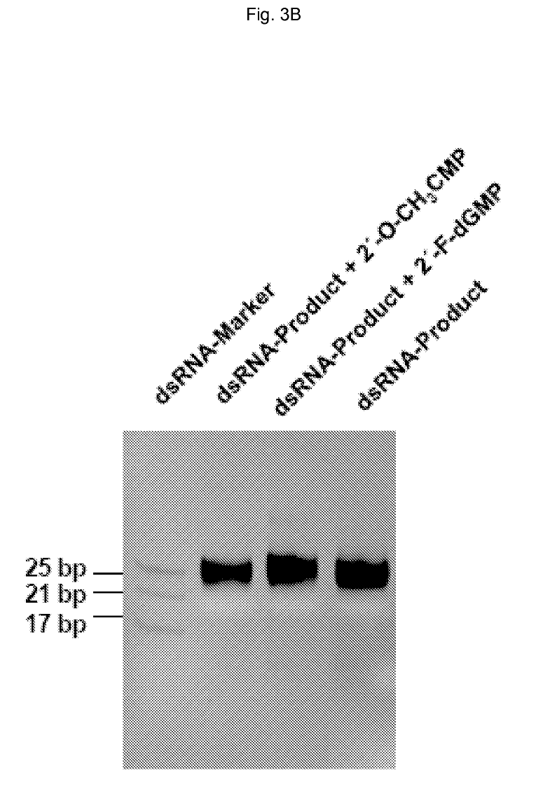

FIG. 3a shows graphical representations of elution profiles resulting in purification of the double-stranded RNA products of the enzymatic synthesis using calicivirus RdRp. The products of a reaction for a normal dsRNA product (upper panel), a dsRNA product including 2-O—CH3—CMP (2′-O-methyl-cytidine-5′-monophosphate, medium panel) and a dsRNA containing 2-F-dGMP (2′-fluoro-2′-deoxy-guanosine-5′-monophosphate) were purified from the reaction mixture by ion exchange chromatography and fractionized.

FIG. 3b shows a photographical representation of a polyacrylamid gel electrophoresis of the fractions indicated (marked with a dashed box) in FIG. 3a. M, molecular size marker for double-stranded RNA (Ambion/Applied Biosystem) as indicated. The product of the synthesis using unmodified nucleotides (dsRNA-product) co-migrates with the 25 bp marker fragment. The product of the synthesis with incorporation of 2′-O-methyl-cydidine-5-triphosphate can also be seen as a band of approximately 25 bp. The same holds true for the product of the synthesis under incorporation of 2′-fluoro-2′-deoxy-guanosine-5′-triphosphate running also as a dsRNA product of around 25 bp.

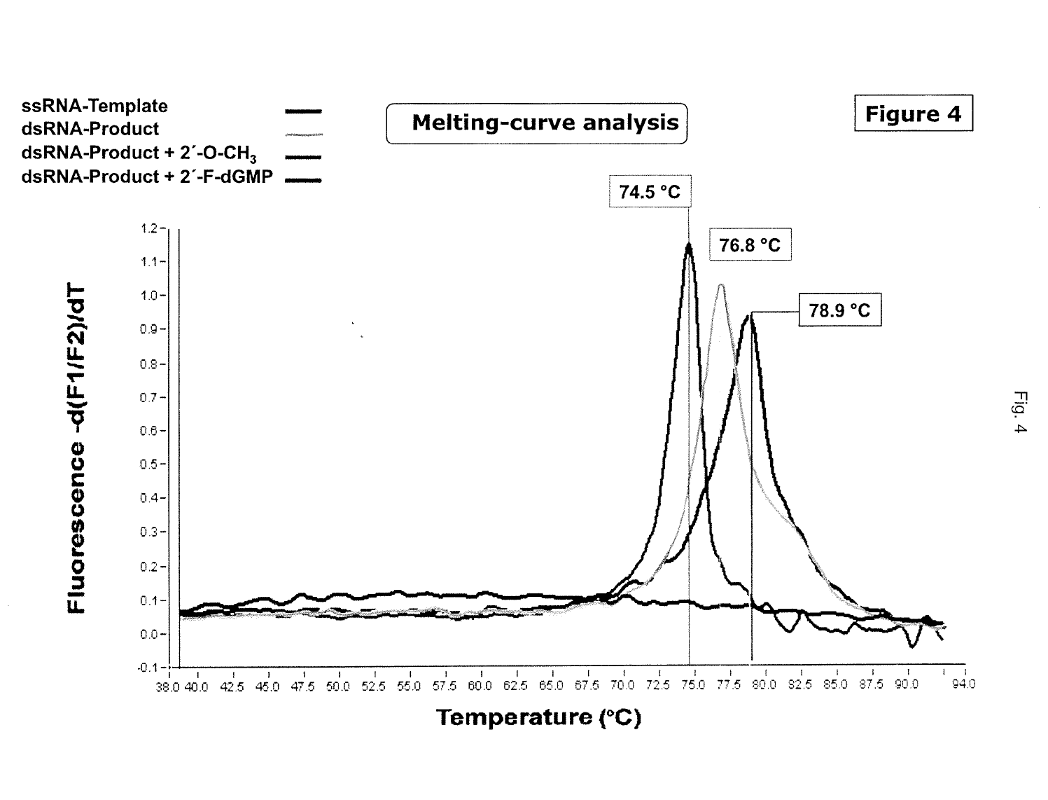

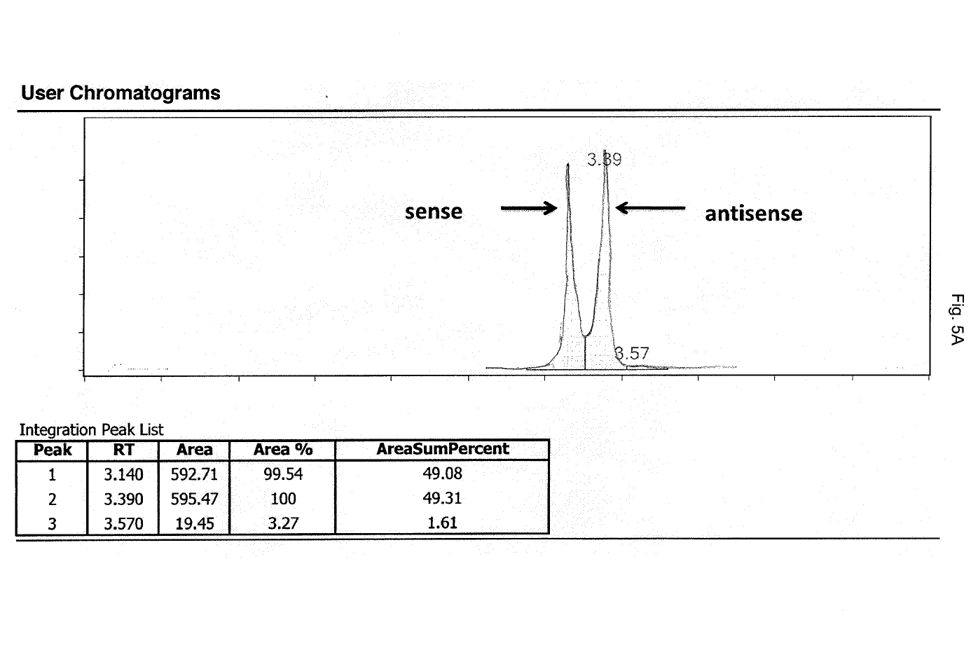

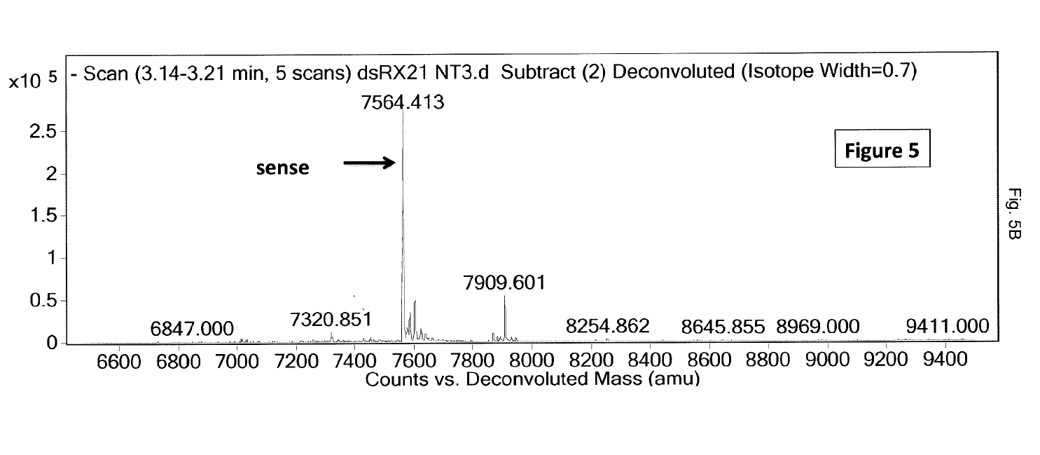

FIG. 4 is a graph showing results of melting point analyses of the products of the enzymatic synthesis by calicivirus RdRp in the absence or presence of modified ribonucleoside triphosphates. The reaction products of a synthesis using unmodified ribonucleotides (dsRNA product), using 2′-O—CH3—CTP (dsRNA product+2-O—CH3—CMP) and using 2′-F-dGTP (dsRNA product+2′F-dGMP) were incubated with SYBR Green I, and the melting points of the products were determined using the LightCycler (Roche diagnostics). The products display different melting points caused by the incorporation of modified nucleotides as compared to unmodified nucleotides.

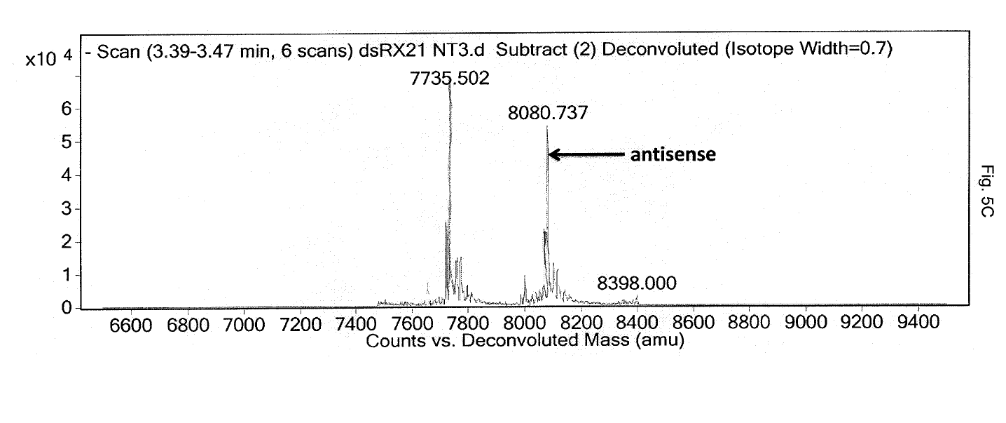

FIG. 5. shows the mass spectrometric analysis of HPLC fractions containing the products of enzymatic synthesis using 2′-O—CH3—CTP (dsRNA product+2-O—CH3—CMP). The masses of the sense strand (template ssRNA, molecular weight of 7564.41) and the antisense strand (synthesized single stranded RNA complementary to the template ssRNA, molecular weight of 8080.73) correspond to the predicted incorporation of 2′-O—CH3—CMP in the antisense strand. A: Graphical representation of the elution profile of the HPLC fractions containing the sense and antisense strand (peaks indicated). B; Graphical representation of the mass spectrometric analysis of the sense strand fraction. C: Graphical representation of the mass spectrometric analysis of the antisense strand fraction.

The following non-limiting examples further illustrate the present invention.

### EXAMPLES

### General Protocol for Enzymatic Synthesis of Modified RNA

The general reaction mixture is as follows:

The reaction mix consists of
RNA-dependent RNA polymerase (RdRp) from calicivurs
Buffer (5-fold): HEPES pH 7.6, 250 mM; MnCl2, 25 mM; DTT 5 mM
Ribonucleoside triphosphates: X, Y, Z, W
Single-stranded RNA (template);

| Sequence: | |
|---|---|
| GCUGAUGCCGUCAAGUUUACCCCC | (SEQ ID NO: 7) |

Aqua. Dest

The reaction mixture is incubated for 2 h at 30 to 42° C. depending on the RdRp used.

X, Y, Z, or W corresponds to one or more of the following ribonucleoside triphosphates or deoxyribonulcleoside triphosphates alone or in combination:

TABLE 1
unmodified and modified nucleotides for X, Y, Z, W

adenosine-5′-triphosphate
cytidine-5-′triphosphate
guanosine-5′-triphosphate
uridine-5′-triphosphate
2′-O-methyl-cytidine-5′-triphosphate
5-aminoallyl-uridine-5′-triphosphate
6-aza-uridine-5′-triphosphate
8-aza-adenosine-5′-triphosphate
5-bromo-uridine-5′-triphosphate
7-deaza-adenosine-5′-triphosphate
7-deaza-guanosine-5′-triphosphate
N⁶-methyl-adenosine-5′-triphosphate
5-methyl-cytidine-5-′triphosphate
pseudo-uridine-5′-triphosphate
4-thio-uridine-5′-triphosphate
2′-amino-2′-deoxy-Uridine-5′-triphosphate
2′-azido-2′-deoxy-uridine-5′-triphosphate
2′-fluoro-2′-deoxy-guanosine-5′-triphosphate
2′-O-methyl-5-Methyl-uridine-5′-triphosphate

### Working Examples 1 to 14 and Comparative Example 15

The reactions mixtures for Examples 1 to 14 and Comparative Example 15 (including only unmodified rNTPs) were set up according to the following Table 2:

TABLE 2
Reaction mixtures
Examples: No. 1 bis 15

| | Reaction Mixture No. | | | | | | | | | | | | | | | |
|---|---|---|---|---|---|---|---|---|---|---|---|---|---|---|---|---|
| Reagent | 1 | | 2 | | 3 | | 4 | | 5 | | 6 | | 7 | | 8 | |
| Buffer (5-fold | 10 | μl | 10 | μl | 10 | μl | 10 | μl | 10 | μl | 10 | μl | 10 | μl | 10 | μl |
| concentrate) | | | | | | | | | | | | | | | | |
| rATP (10 mM) | 2 | μl | 2 | μl | 2 | μl | — | | 2 | μl | — | | — | | 2 | μl |
| rGTP (10 mM) | 2 | μl | 2 | μl | 2 | μl | 2 | μl | 2 | μl | 2 | μl | 2 | μl | 2 | μl |
| rCTP (10 mM) | — | | 2 | μl | 2 | μl | 2 | μl | 2 | μl | 2 | μl | 2 | μl | — | |
| rUTP (10 mM) | 2 | μl | — | | — | | 2 | μl | — | | 2 | μl | 2 | μl | 2 | μl |
| 2′-O-Methyl-C | 2 | μl | — | | — | | — | | — | | — | | — | | — | |
| 5-Aminoallyl-U | — | | 2 | μl | — | | — | | — | | — | | — | | — | |
| 6-Aza-U | — | | — | | 2 | μl | — | | — | | — | | — | | — | |
| 8-Aza-A | — | | — | | — | | 2 | μl | — | | — | | — | | — | |
| 5-Bromo-U | — | | — | | — | | — | | 2 | μl | — | | — | | — | |
| 7-Deaza-A | — | | — | | — | | — | | — | | 2 | μl | — | | — | |
| N6-Methyl-A | — | | — | | — | | — | | — | | — | | 2 | μl | — | |
| 5-Methyl-C | — | | — | | — | | — | | — | | — | | — | | 2 | μl |
| Pseudo-U | — | | — | | — | | — | | — | | — | | — | | — | |
| 4-Thio-U | — | | — | | — | | — | | — | | — | | — | | — | |
| 2′-Amino-2′-deoxy-U | — | | — | | — | | — | | — | | — | | — | | — | |
| 2′-Azido-2′-deoxy-U | — | | — | | — | | — | | — | | — | | — | | — | |
| 2′-Fluoro-2′-deoxy-G | — | | — | | — | | — | | — | | — | | — | | — | |
| 2-O-Methyl-5-Methyl-U | — | | — | | — | | — | | — | | — | | — | | — | |
| Calicivirus RdRp | 5 | μl | 5 | μl | 5 | μl | 5 | μl | 5 | μl | 5 | μl | 5 | μl | 5 | μl |
| Aqua Dest | 26 | μl | 26 | μl | 26 | μl | 26 | μl | 26 | μl | 26 | μl | 26 | μl | 26 | μl |
| ssRNA-Template | 1 | μl | 1 | μl | 1 | μl | 1 | μl | 1 | μl | 1 | μl | 1 | μl | 1 | μl |

| | Reaction Mixture No. | | | | | | | | | | | | | |
|---|---|---|---|---|---|---|---|---|---|---|---|---|---|---|
| | | | | | | | | | | | | | 15 | |
| | 9 | | 10 | | 11 | | 12 | | 13 | | 14 | | (comperative) | |
| Buffer (5-fold | 10 | μl | 10 | μl | 10 | μl | 10 | μl | 10 | μl | 10 | μl | 10 | μl |
| concentrate) | | | | | | | | | | | | | | |
| rATP (10 mM) | 2 | μl | 2 | μl | 2 | μl | 2 | μl | 2 | μl | 2 | μl | 2 | μl |
| rGTP (10 mM) | 2 | μl | 2 | μl | 2 | μl | 2 | μl | — | | 2 | μl | 2 | μl |
| rCTP (10 mM) | 2 | μl | 2 | μl | 2 | μl | 2 | μl | 2 | μl | 2 | μl | 2 | μl |

| | | | | | | | | | |
|---|---|---|---|---|---|---|---|---|---|
| rUTP (10 mM) | — | — | — | — | 2 | μl | — | 2 | μl |

| 2′-O-Methyl-C | — | | — | — | — | — | — | — |
|---|---|---|---|---|---|---|---|---|
| 5-Aminoallyl-U | — | | — | — | — | — | — | — |
| 6-Aza-U | — | | — | — | — | — | — | — |
| 8-Aza-A | — | | — | — | — | — | — | — |
| 5-Bromo-U | — | | — | — | — | — | — | — |
| 7-Deaza-A | — | | — | — | — | — | — | — |
| N6-Methyl-A | — | | — | — | — | — | — | — |
| 5-Methyl-C | — | | — | — | — | — | — | — |
| Pseudo-U | 2 | μl | — | — | — | — | — | — |

| | | | | | | | | |
|---|---|---|---|---|---|---|---|---|
| 4-Thio-U | — | 2 | μl | — | — | — | — | — |

| | | | | | | | | |
|---|---|---|---|---|---|---|---|---|
| 2′-Amino-2′-deoxy-U | — | — | 2 | μl | — | — | — | — |

| | | | | | | | | |
|---|---|---|---|---|---|---|---|---|
| 2′-Azido-2′-deoxy-U | — | — | — | 2 | μl | — | — | — |

| | | | | | | | | |
|---|---|---|---|---|---|---|---|---|
| 2′-Fluoro-2′-deoxy-G | — | — | — | — | 2 | μl | — | — |

| | | | | | | | | |
|---|---|---|---|---|---|---|---|---|
| 2-O-Methyl-5-Methyl-U | — | — | — | — | — | 2 | μl | — |

| | | | | | | | | | | | | | | |
|---|---|---|---|---|---|---|---|---|---|---|---|---|---|---|
| Calicivirus RdRp | 5 | μl | 5 | μl | 5 | μl | 5 | μl | 5 | μl | 5 | μl | 5 | μl |
| Aqua Dest | 26 | μl | 26 | μl | 26 | μl | 26 | μl | 26 | μl | 26 | μl | 26 | μl |
| ssRNA-Template | 1 | μl | 1 | μl | 1 | μl | 1 | μl | 1 | μl | 1 | μl | 1 | μl |

buffer (5x): HEPES pH 7.6, 250 mM; MnCl2, 25 mM; DTT 5 mM
ssRNA-template (1 μg/ul): heteropolymeric sequence, 19 to 25 nt length
modified and/or unmodified ribonucleoside-triphosphate or deoxyribonulcleoside triphosphate were used as stock solutions of: 10 mM
calicivirus RdRp: sapovirus RdRp (SEQ ID NO: 4) was used from stock solution of 70 μM

For RNA synthesis the reaction mixtures according to Examples 1 to 14 and Comparative Example 15 (no modified nucleotides present) were incubated at 30° C. for 2 h.

The abbreviations used in Table 2 above stand for the following nucleotides as outlined in Table 3.

TABLE 3
List of abbreviations for ribonucleoside triphosphates

| Abbreviation | Chemical Name |
|---|---|
| rATP | adenosine-5′-triphosphate |
| rCTP | cytidine-5′-triphosphate |
| rGTP | guanosine-5′-triphosphate |
| rUTP | uridine-5′-triphosphate |
| 2′-O-Methyl-C | 2′-O-methyl-cytidine-5′-triphosphate |
| 5-Aminoallyl-U | 5-Aminoallyl-Uridine-5′-triphosphate |
| 6-Aza-U | 6-aza-uridine-5-′Triphosphate |
| 8-Aza-A | 8-aza-adenosine-5′-triphosphate |
| 5-Bromo-U | 5-bromo-uridine-5-′triphosphate |
| 7-Deaza-A | 7-deaza-adenosine-5′-triphosphate |
| 7-Deaza-G | 7-deaza-guanosine-5′-triphosphate |
| N6-Methyl-A | N⁶-methyl-adenosine-5′-triphosphate |
| 5-Methyl-C | 5-methyl-cytidine-5′-triphosphate |
| Pseudo-U | pseudo-uridine-5′-triphosphate |
| 4-Thio-U | 4-thio-uridine-5′-triphosphate |
| 2′-Amino-2′-deoxy-U | 2′-amino-2′-deoxy-uridine-5′-triphosphate |
| 2′-Azido-2′-deoxy-U | 2′-azido-2′-deoxy-uridine-5′-triphosphate |
| 2′-Fluoro-2′-deoxy-G | 2′-fluoro-2′-deoxy-guanosine-5′-triphosphate |
| 2-O-Methyl-5-Methyl-U | 2-O-methyl-5-methyl-uridine-5′-triphosphate |

Synthesis of Chemically Modified and Unmodified dsRNA Using Calicivirus RdRp

The reaction mixtures of Examples 1 to 14 and Comparative Example 15 were analysed by PAGE (polyacrylamide gel electrophoresis). Bands were detected by ethidium bromide staining. As shown in FIG. 1, the reaction mixtures of Examples 1 to 14 contain a species co-migrating with a 25 bp fragment of a dsRNA marker (lane M) which is also present in the reaction mixture of Comparative Example 15 in which only unmodified nucleotides were included in the reaction mixture.

These data suggest that RdRp of the Caliciviridae are capable of synthesizing dsRNA from various chemically modified building blocks.

Purification and Characterisation of Chemically Modified and Unmodified dsRNA Synthesised by Calicivirus RdRp

The products of the reaction mixtures of Comparative Example 15 (unmodified dsRNA-Product), Example 1 (dsRNA-Product+2-O—CH3—CMP) and Example 13 (dsRNA-Product+2-F-dGMP) were purified by ion exchange (IEX) chromatography and fractionated. Elution profiles are shown in FIGS. 2 and 3. The dashed lines show the fractions suspected to represent the dsRNA which were pooled and further analysed. The detection of the enzymatically synthesized (backbone-) modified (dsRNA-Product+2-F-dGMP and dsRNA-Product+2-O—CH3—CMP) or unmodified double-stranded RNA (dsRNA-Product) was performed with the pooled fractions by PAGE after staining with ethidium bromide and visualisation by UV-transillumination. For comparison, ssRNA template (ssRNA-Template in FIGS. 2a and 2c) and a marker for dsRNA (lane M in FIG. 2c) were also loaded on the gel on separate lanes. The single-stranded RNA used as template for synthesis is visible as a lower band migrating at the level of the 17 bp band of the marker. The synthesized unmodified double-stranded RNA migrates at the level of the 25 bp band of the marker (dsRNA-Product). The synthesized 2′-O-methyl-cytidine-5′-phosphate backbone-modified double-stranded RNA migrates at the level of the 25 bp band of the marker (FIG. 3b, dsRNA-Product+2-O—CH3—CMP). The synthesized 2′-fluoro-2′-deoxy-guanosine-5′-phosphate backbone-modified double-stranded RNA also migrates at the level of the 25 bp band of the marker (FIG. 3b, dsRNA-Product+2-F-dGMP).

Melting Point Analysis of Chemically Modified and Unmodified dsRNA Synthesised by Calicivirus RdRp

The products of the reaction mixtures of Comparative Example 15 (unmodified dsRNA-Product), Example 1 (dsRNA-Product+2-O—CH3—CMP) and Example 13 (dsRNA-Product+2-F-dGMP) were incubated with a double-stranded RNA-intercalating agent (SYBR Green I) and the melting curves of the double-stranded RNA products were measured using the LightCycler device (Roche Diagnostics). The backbone-modified (dsRNA-Product+2-O—CH3—CMP and dsRNA-Product+2-F-dGMP, as indicated) and unmodified RNA (dsRNA-Product, as indicated) display various melting points as indicated in FIG. 3. These different melting curves result from incorporation of modified nucleotides in the RNA-backbone. Importantly, the single-stranded RNA template does not display a melting curve, being single stranded and hence not able to bind SYBR Green I. The shift in the melting points of the backbone-modified dsRNA products to the unmodified comparative dsRNA demonstrated that the RdRp has factually incorporated the modified nucleotides into the dsRNA.

### Mass Spectrometric Characterisation of an Enzymatically Synthesized Double Stranded RNA Having 2′-O—CH3—CMP Incorporation.

The products of the reaction mixture of enzymatic synthesis using 2′-O—CH₃—CTP (dsRNA product+2-O—CH₃—CMP; Example 1) were analyzed by HPLC, with subsequent denaturation of the double stranded RNA leading to elution of the sense (template ssRNA) and antisense (complementary ssRNA) strands. The masses of the sense (template ssRNA, molecular weight of 7564.41) and the antisense (synthesized single stranded RNA complementary to the template ssRNA, molecular weight of 8080.73) were measured by ESI/MS. They were found to correspond exactly to the predicted mass of the template ssRNA and the incorporation of 2′-β-CH₃—CMP in the antisense strand (FIGS. 5B and 5C).

Sequences of the sense (template) and antisense (product) strand:

ssRX21 for:

| | |
|---|---|
| GCUGAUGCCGUCAAGUUUACCCCC | (SEQ ID NO: 7) |
| ssRX21 rev: | |
| 5-P₃-GGGGGUAAACUUGACGGCAUCAGC | (SEQ ID NO: 8) |

C residues in bold mark the 2′-O—CH₃—CMP residues.

Calculated molecular weights (Da): ssRX21 for: 7564,6; ssRX21 rev: 8080,9

MS-Analysis was carried out by LCMS using a standard HPLC system from Agilent Technologies, Inc. (Santa Clara, Calif., USA), equipped with degasser, binary pump, temperature controllable autosampler, column oven and a DAD detector. The effluent from the DAD detector was coupled on-line with an accurate mass Q-TOF mass analyzer which was equipped with a dual spray electrospray source. An absolute amount of 10 mOD of total oligo was injected onto a reversed phase chromatography column and the oligonucleotides were separated using a methanol gradient.

1. A method for enzymatically synthesizing chemically modified RNA comprising the steps of:
(a) providing ssRNA template; and
(b) contacting the ssRNA template with a protein having RNA-dependent RNA polymerase (RdRp) activity under conditions sufficient for RNA synthesis in the presence of at least one ribonucleoside triphosphate having chemical modification at the ribose, phosphate and/or base moiety to provide chemically modified double-stranded RNA (dsRNA),
wherein the chemical modification does not result in labelling of the dsRNA with a radioactive, fluorescent and/or chemical label used for detection of said dsRNA.

2. The method of claim 1 further comprising
(c) separating the dsRNA into ssRNA; and optionally performing the following steps (d) to (f):
(d) repeating steps (b) and (c) n times with n being an integer of at least 1;
(e) carrying out a final step (b); and
(f) stopping the reaction.

3. The method of claim 2 wherein n is an integer from 15 to 40.

4. The method of claim 2 wherein step (c) is carried out by heat denaturation, chemical denaturation or enzymatically.

5. The method of claim 4 wherein the dsRNA is separated into ssRNA by a helicase.

6. The method according to claim 1, wherein the protein having RdRp activity has a “right hand conformation” and the amino acid sequence of the protein comprises the following sequence motifs:
a. XXDYS
b. GXPSG
c. YGDD
d. XXYGL
e. XXXXFLXRXX
with the following meanings:
D: aspartate
Y: tyrosine
S: serine
G: glycine
P: proline
L: leucine
F: phenylalanine
R: arginine
X: any amino acid.

7. The method of claim 6 wherein the protein is an RNA-dependent RNA polymerase of a virus of the Caliciviridae family.

8. The method of claim 7 wherein the protein is an RNA-dependent RNA polymerase of a norovirus, sapovirus, vesivirus or lagovirus.

9. The method of claim 8 wherein the protein is selected from the group consisting of an RNA-dependent RNA polymerase of the norovirus strain (HuCV/NL/Dresden174/1997/GE (GenBank acc. No. AY741811), an RNA-dependent RNA polymerase of the sapovirus strain pJG-Sap01 (GenBank acc. No. AY694184), and an RNA-dependent RNA polymerase of the vesivirus strain FCV/Dresden/2006/GE (GenBank acc. No DQ424892).

10. The method of claim 8 wherein the protein has an amino acid sequence selected from the group consisting of SEQ ID NO: 1, SEQ ID NO: 2, SEQ ID NO: 3, SEQ ID NO: 4, SEQ ID NO 5; and SEQ ID NO: 6.

11. The method according to claim 1, wherein the ssRNA template has a length of 15 to 30, preferably 21 to 28 nucleotides, more preferably 21 to 23 nucleotides.

12. The method according to claim 1, wherein the chemically modified dsRNA has an increased stability as compared to the non-modified analogue.

13. The method according to claim 1, wherein the at least one ribonucleoside triphosphate has a chemical modification at the ribose moiety.

14. The method of claim 13 wherein the 2′-OH group of the ribose moiety is replaced by a group selected from H, OR, R, halo, SH, SR, NH₂, NHR, NR₂ or CN, wherein R is C₁-C₆ alkyl, alkenyl or alkynyl and halo is F, CI, Br or I.

15. The method of claim 14 wherein the at least one chemically modified ribonucleoside triphosphate is selected from the group consisting of 2′-O-methyl-cytidine-5′-triphosphate, 2′-amino-2′-deoxy-uridine, 2′-azido-2′-deoxy-uridine-5′-triphosphate, 2′-fluoro-2′-deoxy-guanosine-5′-triphosphate and 2′-O-methyl-5-methyl-uridine-5′-triphosphate.

16. The method according to claim 1, wherein the at least one chemically modified ribonucleoside triphosphate has a chemical modification at the base moeity.

17. The method of claim 16 wherein the at least one chemically modified ribonucleoside triphosphate is selected from the group consisting of 5-aminoallyl-uridine-5′-triphosphate, 6-aza-uridine-5′-triphosphate, 8-aza-adenosine-5′-triphosphate, 5-bromo-uridine-5′-triphosphate, 7-deaza-adenosine-5′-triphosphate, 7-deaza-guanosine-5′-triphosphate, N⁶-methyladenosine-5′-triphosphate, 5-methyl-cytidine-5-triphosphate, pseudo-uridine-5′-triphosphate, 4-thio-uridine-5′-triphosphate.

18. The method according to claim 1, wherein the at least one chemically modified ribonucleotide has a chemical modification at the phosphate moiety.

19. The method of claim 18 wherein the at least one chemically modified ribonucleoside triphosphate is a phosphothioate analogue.

20. A kit for carrying out the method according to claim 1 comprising:
a protein having RdRp activity, preferably a RdRp of virus of the Caliciviridae family;
a buffer for providing conditions sufficient for RNA synthesis by the protein having RdRp activity;
rATP, rGTP, rCTP, rUTP;
at least one ribonucleoside triphosphate having chemical modification at the ribose, phosphate and/or base moiety wherein the chemical modification does not provide a label for radioactive, fluorescent or chemical detection;
optionally, stop solution; and
optionally, a helicase.
